(19) United States
(12) Patent Application Publication
(10) Pub. No.: US 2024/0376554 A1
(43) Pub. Date: Nov. 14, 2024

(54) METHOD OF ASSESSING BACTERIAL VIABILITY AND BACTERIAL COMMUNITY STRUCTURE CHANGES

(71) Applicant: The Trustees of Princeton University, Princeton, NJ (US)

(72) Inventors: Ellen ACOSTA, Princeton, NJ (US); Zemer GITAI, Princeton, NJ (US)

(73) Assignee: The Trustees of Princeton University, Princeton, NJ (US)

(21) Appl. No.: 18/682,168

(22) PCT Filed: Aug. 11, 2022
(86) PCT No.: PCT/US2022/040073

§ 371 Date: Feb. 8, 2024

Related U.S. Application Data
(60) Provisional application No. 63/232,921, filed on Aug. 13, 2021.

(51) Int. Cl. C12Q 1/689; C12Q 1/6806; C12Q 1/6818; C12Q 1/6851; C12Q 1/6874

(52) U.S. Cl. CPC C12Q 1/689; C12Q 1/6818; C12Q 1/6851; C12Q 1/6806; C12Q 1/6874

(57) ABSTRACT

A highly reliable and accurate method of assessing bacterial community viability has been developed that allows for the assessment of extremely low biomass samples, which cannot be done with traditional methods, such as qPCR. The method utilizes both PMA and droplet digital PCR (PMA-ddPCR), resulting in very accurate quantification of DNA even at very low abundances. Comparing DNA abundance in untreated samples to DNA abundance in PMA-treated samples allows the calculation of the overall viability of bacteria in any given sample. Further, PMA can be combined with traditional RNA gene sequencing (using gene-specific primers for a target species or strain) to accurately profile, e.g., the human skin microbiome, which has previously been done using traditional sequencing methods alone, but this method allows for a species-level understanding of the viable (and nonviable) components of any complex bacterial community.

### CROSS-REFERENCE TO RELATED APPLICATIONS

This application claimed priority to U.S. Provisional Patent Application No. 63/232,921 filed on Aug. 13, 2021, the entirety of which is incorporated by reference herein.

### STATEMENT REGARDING FEDERALLY SPONSORED RESEARCH OR DEVELOPMENT

This invention was made with government support under Grant Nos. AI124669 and GM007388 awarded by the National Institutes of Health. The government has certain rights in the invention.

### BACKGROUND

The ability to quantify living cells (e.g., bacterial cells) is important to various industries, including the food, beverage, pharmaceutical, and environmental industries, and may need to be determined under various conditions, including under research, clinical, or manufacturing conditions.

Several methods are currently employed by these industries to quantify prokaryotic and eukaryotic cells. These methods include, but are not limited to, the standard plate count, dye reduction and exclusion methods, electrometric techniques, microscopy, and flow cytometry, among others. However, none of these methods provide accurate absolute quantification of bacterial viability.

### BRIEF SUMMARY

In some embodiments, a method for accurately determining the absolute viability of a bacterial sample is provided. The method may include splitting a sample containing a plurality of bacterial cells into a first subsample and a second subsample, where the two subsamples are of equal (or substantially equal) volume. Only the first subsample is treated with propidium monoazide (PMA). Specifically, it may be treated with sufficient PMA to achieve a target concentration. The first subsample is then incubated without exposure to a blue light wavelength for a first period of time (e.g., such as 5-20 minutes). After incubation, the PMA may then be activated and cross-linked by exposing the first subsample to blue light wavelengths for a second period of time (e.g., such as 10-30 minutes). DNA may then be extracted from both the first subsample and the second subsample. In some embodiments, DNA may be extracted using a standard DNA isolation kit. The extracted DNA of each sample is then separately quantified using droplet-digital PCR (ddPCR) and gene-specific primers for a target species or strain. The absolute viability of the original sample is then determined by comparing a value of the quantified DNA from the first sample to a value of the quantified DNA from the second sample.

In some embodiments, the comparing values of quantified DNA may include determining a ratio between the value of the quantified DNA from the first sample to the value of the quantified DNA from the second sample.

In some embodiments, the plurality of bacterial cells may include only a single strain of bacteria. In some embodiments, the plurality of bacterial cells may include only a single species of bacteria. In some embodiments, the plurality of bacterial cells may include a plurality of species of bacteria.

In some embodiments, the sample may be from a human microbiome, or from a built surface. In some embodiments, the sample may include bacteria from human skin, saliva, nasal cavity, or gut. In some embodiments, the sample may include bacteria from surface in a hospital, in a house, or in an office building.

In some embodiments, the gene-specific primers are universal 16S primers. In some embodiments, the gene-specific primers are primers specific to a flagellar gene in Salmonella typhimurium.

In some embodiments, a method for assessing bacterial community structure changes may be provided. This method is very similar to the method described above. The method may include splitting a sample containing a plurality of bacterial cells into a first subsample and a second subsample, where the two subsamples are of equal (or substantially equal) volume. Only the first subsample is treated with propidium monoazide (PMA). Specifically, it may be treated with sufficient PMA to achieve a target concentration. The first subsample is then incubated without exposure to a blue light wavelength for a first period of time (e.g., such as 5-20 minutes). After incubation, the PMA may then be activated and cross-linked by exposing the first subsample to blue light wavelengths for a second period of time (e.g., such as 10-30 minutes). DNA may then be extracted from both the first subsample and the second subsample. In some embodiments, DNA may be extracted using a standard DNA isolation kit. The method may include sequencing the extracted DNA from both the first and second subsamples using gene-specific primers for a target species or strain and determining an abundance of bacterial species in both the first and second subsamples. A bacterial community structure can be assessed by comparing the relative abundance of bacterial species in the first and second subsamples. By comparing structures of samples taken at different points in time, or from different locations, changes to the bacterial community structure can be assessed.

In some embodiments, comparing values of quantified DNA may include determining a ratio between the value of the quantified DNA from the first sample to the value of the quantified DNA from the second sample.

In some embodiments, the sample may be from a human microbiome, or from a built surface. In some embodiments, the sample may include bacteria from human skin, saliva, nasal cavity, or gut. In some embodiments, the sample may include bacteria from surface in a hospital, in a house, or in an office building.

### BRIEF DESCRIPTION OF DRAWINGS

FIGS. 1 and 2 are flowcharts of embodiments of a method.

FIG. 3 is a graph showing PMA-ddPCR validation controls on known ratios of heat-killed and exponentially-growing E. coli cultures.

FIG. 4 is a graph showing a standard curve showing a correlation between PMA-ddPCR counts and CFU for exponentially-growing Staphylococcus epidermidis. The 95% confidence interval is shown in dashed lines.

FIG. 5 is a graph showing PMA-ddPCR and viability scores for human skin microbiomes. Standard curve generated using Staphylococcus epidermidis cultures shown with dashed line representing 95% confidence interval (same as in FIG. 4). Open and closed circles represent skin microbiome samples that did (closed circles) or did not (open circles) receive PMA treatment. Paired samples are connected with a left-facing arrow to show the downward shift in DNA abundance with the inclusion of PMA.

FIG. 6 is a graph showing a comparison of the predicted ddPCR counts to measured ddPCR counts based on CFU for samples that were treated with PMA (closed circles) and samples that were not treated with PMA (open circles) (mean for PMA-treated samples is 1.31, mean for untreated samples is 82.2).

FIG. 7 is a graph showing a comparison of ddPCR-based viability scores to CFU-based viability score (mean=0.93).

FIG. 8 is a graph showing PMA-ddPCR on skin and non-skin microbiome sites shows that the viability score of the skin microbiome is significantly lower than other microbiome sites. **** indicates P≤0.0001 for Student's T Test on pooled skin and non-skin samples.

FIG. 9 is a graph showing relative abundance in hair shafts for the top three most abundant (overall) bacterial taxa as assessed by traditional sequencing and PMA-seq.

FIG. 10 is a graph showing a viability score for three skin sites with and without use of lysostaphin, and using either 16S primers or Staphylococcus-specific PCR primers. The three skin sites with the most abundant bacterial DNA are shown. Half of each sample was treated with lysostaphin prior to DNA isolation to assess how the viability score would change. ddPCR was performed on each sample using either 16S primers (white bars) or Staphylococcus-specific (black bars) primers. The dashed line indicates the average viability score of non-skin microbiome sites (0.66). N=3 for all.

### DETAILED DESCRIPTION

To avoid the inaccuracies of conventional techniques, a method is needed that can take sample and give an accurate, absolute score of what fractions of bacteria in the sample is live and/or dead, even if the amount of bacteria is at relatively low levels.

For example, when testing low biomass samples, conventional techniques tend to have issues with low DNA signal-to-noise ratios.

When using “quantitative PCR” (qPCR) to determine cell viability, the results are more qualitative than quantitative. The output from qPCR is a CT number, which is the number of cycles it required before the system could achieve a detectable level of DNA. The higher the CT number, the more cycles it took, and the more cycles it took, the less DNA was present in the initial sample. The results are non-linear, and CT number is inversely proportional to concentration, making it impossible to convert CT number to an absolute number with any degree of accuracy.

Thus, a method for providing accurate absolute quantification of bacterial viability is desirable and useful.

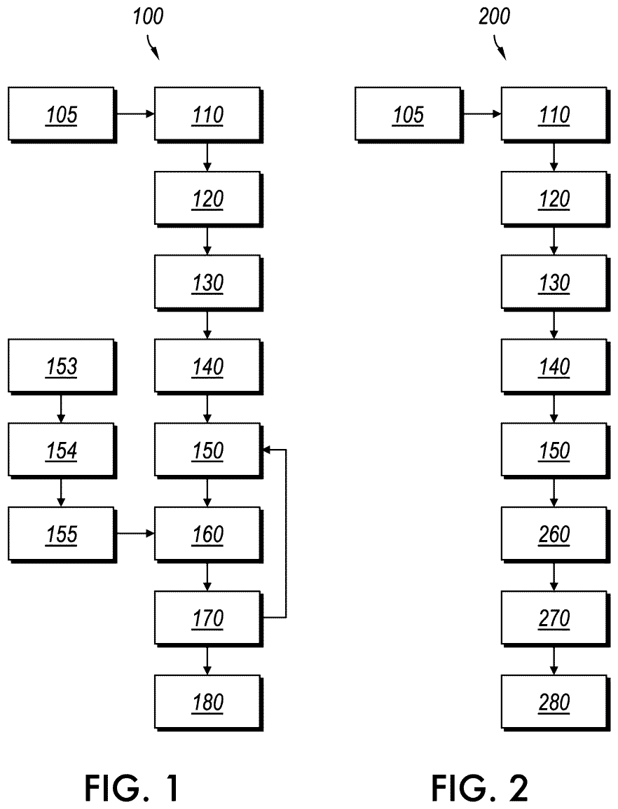

Referring to FIG. 1, in some embodiments, a method 100 for accurately determining the absolute viability of a bacterial sample is provided.

The method may include gather, collecting, and/or providing 105 a sample containing a plurality of bacterial cells. In some embodiments, the sample may be from a human microbiome, or from a constructed or built surface (e.g., a table, cabinet, door handle, etc.). In some embodiments, the sample may include bacteria from human skin, saliva, nasal cavity, or gut. In some embodiments, the sample may include bacteria from surface in a hospital, in a house, or in an office building. In some embodiments, the sample may be gathered by a doctor, nurse, or technician. In some embodiments, the sample may be gathered by a patient, subject, or user. In some embodiments, the samples are collected in appropriate sterile sample containers.

In some embodiments, the plurality of bacterial cells may include only a single strain of bacteria. In some embodiments, the plurality of bacterial cells may include only a single species of bacteria. In some embodiments, the plurality of bacterial cells may include a plurality of species of bacteria.

The method may include splitting 110 the sample into two subsamples—a first subsample and a second subsample, where the two subsamples are of substantially equal volume. As used herein, “substantially equal volume” refers to volumes that are ±5% from each other.

The method may include treating 120 only the first subsample with propidium monoazide (PMA). In some embodiments, sufficient PMA is added to achieve target concentration. In some embodiments, the target concentration of PMA is 5-100 μM in the subsample.

The first subsample may then be incubated 130 without exposure to blue light wavelengths for a first period of time (blue light wavelengths may include wavelengths of about 415-495 nm). In some embodiments, the amount of time is at least 5 minutes. In some embodiments, the amount of time is less than 60 minutes. In some embodiments, the amount of time is 5-20 minutes.

The PMA may then be activated and cross-linked by exposing 140 the first subsample to one or more blue light wavelengths for a second period of time. In some embodiments, the amount of time is at least 10 minutes. In some embodiments, the amount of time is less than 60 minutes. In some embodiments, the amount of time is 10-30 minutes.

DNA may then be extracted 150 from both the first subsample and the second subsample. In some embodiments, DNA may be extracted using a standard DNA isolation kit.

The extracted DNA of each sample is then separately quantified 160 using droplet-digital PCR (ddPCR) and gene-specific primers for a target species or strain.

The gene-specific primers may be selected based on, e.g., the desired level of specificity, e.g., the desired level of specificity required in the analysis. For example,

In some embodiments, the gene-specific primers are specific to only one target strain in the sample. In some embodiments, the gene-specific primers are specific to only one target species in the sample. In some embodiments, the gene-specific primers are expected to be specific to all or substantially all species in a target genus in the sample (e.g., all Escherichia in the sample).

In some embodiments, the gene-specific primer targets a gene in one or more gram-positive species. In some embodiments, the gene-specific primer targets a gene in one or more gram-negative species.

In some embodiments, the gene-specific primers are universal 16S primers. Such primers are well known in the art.

In some embodiments, the gene-specific primers are primers specific to a flagellar gene in Salmonella typhimurium. Such primers are known in the art.

In some embodiments, the method may include providing 155 (at some point prior to the quantification step) the appropriate gene-specific primers for the quantification. In some embodiments, the method may include identifying 153 at least one target gene that is present in at least one strain or species in the sample, and not present in at least one other strain or species. In some embodiments, the method may include designing 154 the appropriate gene-specific primers, using known methods, that targets the identified gene or genes.

The absolute viability of the original sample is then determined by comparing 170 a value of the quantified DNA from the first sample to a value of the quantified DNA from the second sample.

In some embodiments, the comparing values of quantified DNA may include determining a ratio between the value of the quantified DNA from the first sample to the value of the quantified DNA from the second sample.

In some embodiments, multiple ddPCR quantifications can be performed, each resulting in a viability score. For example, as shown in FIG. 1, in some embodiments, steps 150, 160, and 170 may be repeated, using, e.g., different primers, to assess different bacteria in the sample.

A bacterial community structure can be assessed by considering one or more of these ddPCR quantifications taken from the same set of samples, and determination of absolute viabilities. For example, if three different quantifications are performed to consider three different species in a sample, the bacterial community structure may be a compilation of all three viability scores.

In some embodiments, changes in a bacterial community structure may be assessed 180 by comparing two different bacterial community structures. In some embodiments, a bacterial community structure from sample gathered from a source at a first point in time is compared to bacterial community structure gathered from the source at a second (later) point in time. For example, in some embodiments, it may be useful to compare a sample from a human microbiome gathered before a treatment to a sample from the same microbiome gathered after the treatment.

In some embodiments, a bacterial community structure from sample gathered from a first source is compared to bacterial community structure gathered from a second source. For example, it may be useful to understand how a bacterial community structure of a sample from a hospital chair, table, or bed differs from a sample from a different element or component (e.g., a drawer handle or doorknob) in the same room as the chair, table, or bed.

Referring to FIG. 2, in some embodiments, a method 200 for assessing bacterial community structure changes may be provided.

The method may include gather, collecting, and/or providing 105 a sample containing a plurality of bacterial cells.

In some embodiments, the sample may be from a human microbiome, or from a constructed or built surface (e.g., a table, cabinet, door handle, etc.). In some embodiments, the sample may include bacteria from human skin, saliva, nasal cavity, or gut. In some embodiments, the sample may include bacteria from surface in a hospital, in a house, or in an office building. In some embodiments, the sample may be gathered by a doctor, nurse, or technician. In some embodiments, the sample may be gathered by a patient, subject, or user. In some embodiments, the samples are collected in appropriate sterile sample containers.

In some embodiments, the plurality of bacterial cells may include only a single species of bacteria. In some embodiments, the plurality of bacterial cells may include a plurality of species of bacteria.

The method may include splitting 110 the sample into two subsamples—a first subsample and a second subsample, where the two subsamples are of substantially equal volume. As used herein, “substantially equal volume” refers to volumes that are ±5% from each other.

The method may include treating 120 only the first subsample with propidium monoazide (PMA). In some embodiments, sufficient PMA is added to achieve target concentration. In some embodiments, the target concentration of PMA is 5-100 μM in the subsample.

The first subsample may then be incubated 130 without exposure to blue light wavelengths for a first period of time. In some embodiments, the amount of time is at least 5 minutes. In some embodiments, the amount of time is less than 60 minutes. In some embodiments, the amount of time is 5-20 minutes.

The PMA may then be activated and cross-linked by exposing 140 the first subsample to blue light wavelengths for a second period of time. In some embodiments, the amount of time is at least 10 minutes. In some embodiments, the amount of time is less than 60 minutes. In some embodiments, the amount of time is 10-30 minutes.

DNA may then be extracted 150 from both the first subsample and the second subsample. In some embodiments, DNA may be extracted using a standard DNA isolation kit.

In some embodiments, the, method may include sequencing 260 the extracted DNA from both the first and second subsamples using gene-specific primers for a target species or strain and determining an abundance of bacterial species in both the first and second subsamples.

In some embodiments, comparing values of quantified DNA may include determining a ratio between the value of the quantified DNA from the first sample to the value of the quantified DNA from the second sample.

A bacterial community structure may then be assessed 270 by comparing the relative abundance of bacterial species in the first and second subsamples. As disclosed previously, by comparing structures of samples taken at different points in time, or from different locations, changes to the bacterial community structure can be assessed.

### Example

DNA sequencing of samples collected by sterile swabbing is the most common method used to evaluate the skin microbiome because it is simple, noninvasive, and has been shown to result in higher consistency than other sampling methods. However, few bacterial cells were observed on the skin surface in this example, it was questioned whether the bacterial DNA that is present on the skin surface and accessible for sampling by swabs is representative of the viable microbiome. Thus, it was necessary to implement a method that allowed the quantification and comparison of total bacterial DNA with bacterial DNA.

Specifically, the cell-impermeable small molecule propidium monoazide (PMA) was utilized, which binds irreversibly to double-stranded DNA upon photoactivation to inhibit PCR amplification. When PMA photoactivation is performed before the cell lysis step of DNA isolation, the genomic DNA inside viable bacteria is protected from PMA binding because PMA is cell impermeable, while cell-free DNA or DNA within permeabilized bacteria becomes PMA-bound. Thus, by comparing DNA quantities in samples with and without PMA treatment, an assessment of the viability of a bacterial population is enabled.

To quantify bacterial DNA and assess the viability of a population of cells, the implemented method combined the use of PMA with droplet digital PCR (PMA-ddPCR).

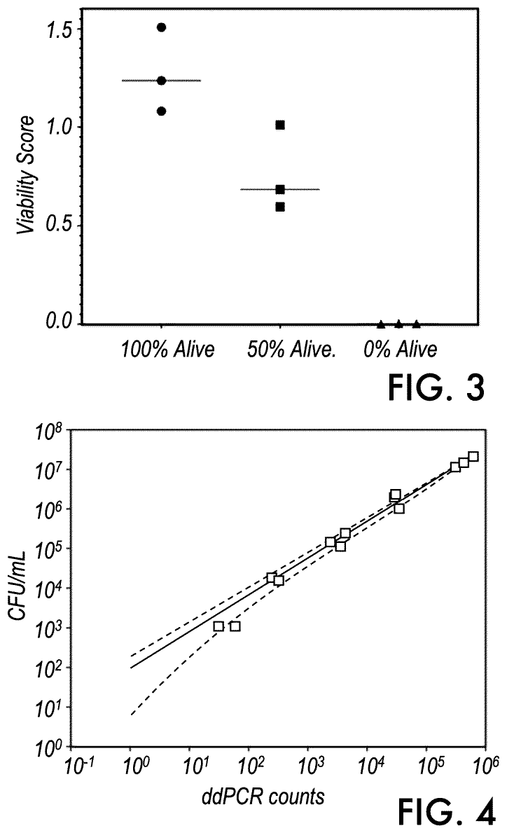

To assure that PMA-ddPCR would enable the ability to reliably gauge the fraction of viable cells in a population, the method was first validated with regards to ensuring it generated the expected results using known ratios of heat-killed and exponentially-growing E. coli cultures. See FIG. 3. Known ratios of live and heat-killed E. coli cultures were mixed and subjected to PMA treatment. First, an overnight culture of E. coli NCM3722 was back-diluted into fresh LB media at a ratio of 1:1000 and grown at 37° C. for 4 hours to reach mid-log. Half of the exponentially-growing culture was heat-killed by incubating at 70° C. for 20 minutes while the other half remained at room temperature. The heat-killed E. coli cultures were allowed to cool to room temperature before combining with non-heat-killed E. coli cultures to achieve a 50% (by volume) heat-killed mixture. For the 0% heat-killed, no heat-killed bacteria were added. Likewise, for the 100% heat-killed, only heat-killed bacteria were used. Each condition was mixed well and then split evenly between two sterile 1.5 mL microcentrifuge tubes.

The PMA treatment used in this example includes, after collection, samples were split evenly between two sterile 1.5 mL microcentrifuge tubes. PMA (Biotium Inc.) was added to one of the two tubes to a final concentration of 50 μM. All tubes were incubated in the dark at room temperature for 10 mins before being exposed to light to cross-link PMA molecules using the PMA-Lite™ LED Photolysis Device (Biotium Inc.). DNA was then isolated from all samples using the DNeasy PowerSoil Kit (Qiagen). If lysostaphin (Sigma-Aldrich) was used, it was added following PMA activation and before DNA isolation to a final concentration of 0.1 mg/mL and incubated at room temperature for 30 mins.

The Bio Rad QX200 AutoDG Droplet Digital PCR System was used to quantify extracted DNA from microbiome samples and from pure bacterial cultures. Reaction mixtures contained 2× QX200 ddPCR EvaGreen Supermix and universal 16S qPCR primers at 10 nM concentrations in a total volume of 25 μL. Primer sequences can be found in Table S1. Reaction mixtures were transferred to sterile ddPCR 96-well plates (BioRad #12001925) which were loaded into the QX200 Automated Droplet Generator. After 476 droplet generation, the plate was heat-sealed using the PX1 PCR Plate Sealer (BioRad #1814000) and PCR was performed with a pre-step of 95 C for 5 minutes followed by 40 rounds of amplification with 60 C, 1-minute extensions and a final hold temperature of 12 C using a C1000 Touch Thermal Cycler (BioRad #1851197). Samples were subsequently loaded into the QX200 Droplet Reader for quantification. Automatic thresholding was performed using the Quantasoft software and subsequently exported to Microsoft Excel for analysis. Significance was calculated using a Student's T-Test. To calculate the viability score for a given pair of “−PMA” and “+PMA” matched samples, the following calculation was done: copies per 20 μL without PMA/copies per 20 μL with PMA.

Referring to FIG. 3, it should be noted that PMA-ddPCR performed on a population of exponentially growing cells resulted in a viability score slightly above 1.0 (1.23), which was likely due to continued bacterial growth during the experiment. PMA-ddPCR performed on a population of 100% heat-killed cells resulted in a viability score of 0. Populations consisting of 50% (by volume) heat-killed and 50% exponentially growing cells exhibited an average viability score of 0.69.

As a further demonstration that PMA-ddPCR provides an accurate measure of DNA from viable cells, it was sought to determine whether PMA-ddPCR accurately approximated the number of culturable bacteria. To this end, serial dilutions of a skin-resident bacterial species, Staphylococcus epidermidis, were used, and a standard curve correlating DNA abundance was generated (quantified by PMA83 ddPCR) and CFU/mL (quantified by classical plating). Cultures of S. epidermidis EGM 2-06 were grown in tryptic soy broth (TSB) overnight and diluted 1:1000 the following morning in TSB and grown for 4 hours until a final OD of 0.4. Tenfold dilutions of S. epidermidis culture were then prepared, plated for CFU on 5% sheep blood in tryptic soy agar (VWR International) and divided between two 1.5 mL microcentrifuge tubes. PMA was added to one tube for a final concentration of 50 μM and the other tube was left untreated. PMA activation and DNA isolation was then done according to the methods outlined above.

It was found that the PMA-ddPCR and CFU/mL values were highly correlated. See FIG. 4.

Performing ddPCR without the use of PMA on an exponentially-growing population of S. epidermidis yielded similar results, indicating both that the PMA treatment itself does not significantly alter viability and that an exponentially-growing culture of bacteria is comprised of mostly viable cells. Together, these controls confirm that PMA-ddPCR represents a good proxy for the amount of DNA in a sample present within intact bacteria.

Next, human skin microbiome samples were collected using 90 sterile swabbing technique to determine if the findings with in-vitro bacterial cultures extend to other microbiomes, such as skin microbiome samples.

Participants were healthy volunteers, aged 26-35, with no history of chronic skin conditions or autoimmune disease and were not using antibiotics. Skin microbiomes from healthy volunteers were collected using sterile foam-tipped collection swabs pre-moistened with sterile 1× DPBS. Detergent in the swabbing buffer was not used in order to avoid negatively affecting bacterial cell membranes and altering viability scores. Areas of interest were sampled for 60 seconds before being re-suspended in sterile 1× DPBS. Hair shaft samples were collected by plucking hairs and using only the bulb portion. Human skin microbiome samples were plated for CFU calculations prior to the addition of PMA. Samples were plated on blood agar plates (5% sheep blood in tryptic soy agar, VWR International) and grown for 24-48 hours aerobically or anaerobically.

To quantify the number of viable bacteria directly, a small amount of each sample was plated using the standard conditions for culturing skin microbes (5% sheep blood in tryptic soy agarose plates incubated both aerobically and anaerobically).

To determine whether PMA-ddPCR or traditional ddPCR better represented the number of viable skin microbiome bacteria, each sample was split into two equal halves and one half was treated with PMA as disclosed herein prior to DNA isolation, leaving the other half untreated.

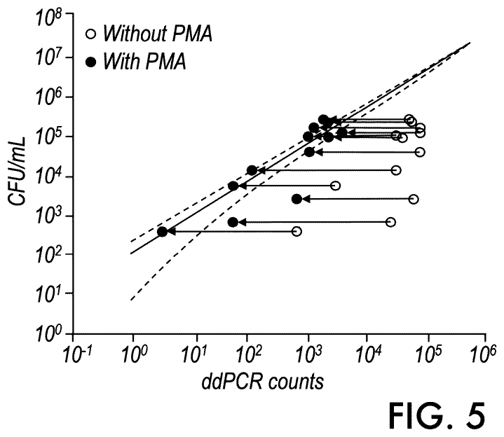

We quantified bacterial DNA in both samples by ddPCR using universal bacterial 16S primers. We found that for each sample, the PMA-ddPCR quantification closely matched the standard curve generated with S. epidermidis, suggesting that the use of PMA allows for an accurate quantification of viable bacterial DNA. See FIG. 5.

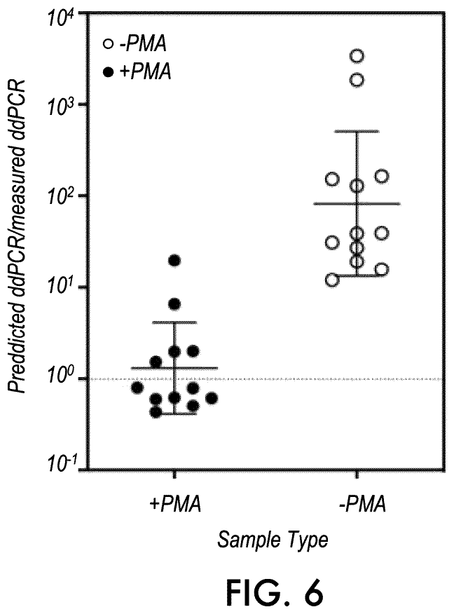

More specifically, quantifying the bacterial DNA in skin microbiome samples without the use of PMA resulted in DNA quantities that were, on average, 82 times higher than predicted by the standard curve, while the use of PMA brought this value down to just 1.3. See FIG. 6.

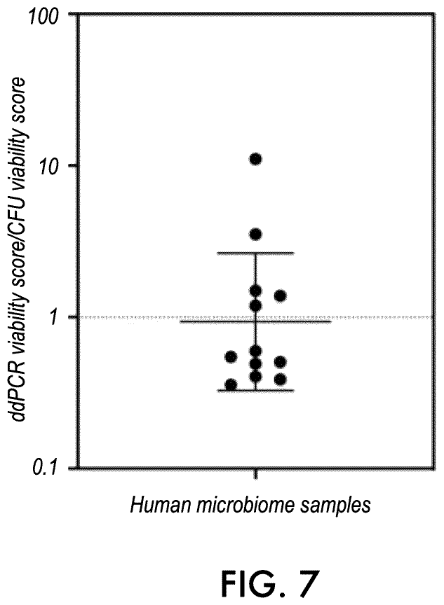

Using ddPCR counts to predict CFU showed similar results, as ddPCR in the absence of PMA yielded values that predicted CFU counts 58.5 times greater than those measured, while PMA-ddPCR yielded values that predicted CFU counts that were on average only 1.28 times greater than the actual cultured CFU. Calculating the ratio of ddPCR counts between samples without PMA and samples with PMA allowed us to generate a viability score for any given microbiome sample. Additionally, we calculated a CFU-based viability score by comparing the CFU predicted by the ddPCR counts in a sample without the use of PMA to the actual CFU. Using either the ddPCR-based method or the CFU-based method resulted in similar viability scores and allowed us to gauge the overall fraction of viable bacteria in a population. See FIG. 7.

The viability scores (using the PMA-ddPCR-based method) of DNA sampled from different skin microbiome sites were then evaluated by swabbing the skin of four healthy human volunteers at eight sites. Skin was swabbed at sebaceous sites (lower back, glabella, hair shaft, retroauricular crease), moist sites (antecubital fossa, nares, popliteal fossa), and dry sites (dorsal forearm). PMA-ddPCR revealed that the viability scores for these sites ranged between 0.02 and 0.12 (0 represents a fully-nonviable population, 1.0 represents a fully-viable population), indicating that the majority of bacterial DNA found on the skin surface is not associated with viable cells.

To investigate whether this was a skin-specific phenomenon, several non-skin microbiome sites (tongue, saliva, plaque, and feces) were tested. Tongue microbiome samples were collected using sterile foam-tipped collection swabs. Saliva was collected in sterile 50 mL conicals from healthy volunteers. Plaque was collected by scraping the teeth of healthy volunteers using sterile toothpicks and re-suspending the collection in sterile 1× DPBS. The human fecal sample was collected in sterile collection containers.

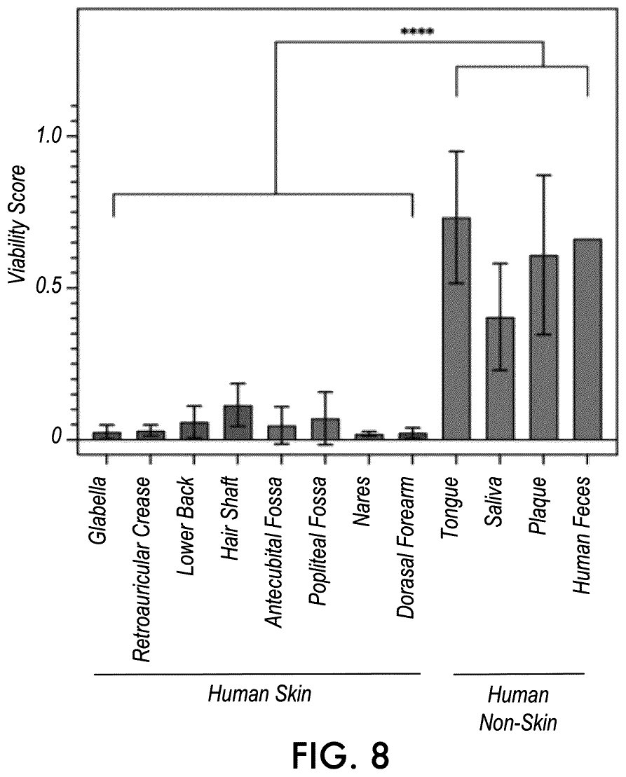

It was found that in all non-skin microbiome sites, the viability score was significantly higher than for the skin, ranging from 0.4 (saliva) to 0.87 (feces). See FIG. 8. These results provide independent support that the skin surface is populated by few viable bacterial cells, indicating that the surface of healthy non-sterilized human skin is sparsely colonized. While these data indicate that bacterial DNA on the skin surface is predominantly not associated with viable bacterial cells, the results do not suggest that there are no viable cells on the skin surface. Rather, the data indicates that the majority of the bacterial on the skin surface is not in intact bacteria such that specifically assessing the DNA associated with intact cells using PMA provides a much more accurate estimation of the viable skin microbiome.

Like many microbiomes, the existing knowledge of the skin microbiome is heavily based upon bacterial 16S rRNA gene amplicon sequencing, which was developed to assess bacterial populations while avoiding biases introduced by culturing methods. However, the findings suggest that using 16S rRNA gene amplicon sequencing to study the skin microbiome is not entirely unbiased, as most of the DNA in these samples is from nonviable bacteria and traditional rRNA gene amplicon sequencing does not differentiate between DNA originating from live or dead cells. The inability of 16S rRNA gene amplicon sequencing to differentiate between these two types of bacterial populations has been mentioned as a potential downfall of the method, but has not been addressed (8).

To evaluate how accurately traditional 16S rRNA gene amplicon sequencing captures the living skin microbiome composition, PMA followed by 16S rRNA gene amplicon sequencing (PMA-seq) was utilized.

DNA was isolated from microbiome samples (with and without 497 PMA) using the DNeasy PowerSoil Kit (Qiagen). The V1-V3 region of the 16S gene was amplified using the primers 27 F 534R. Illumina sequencing libraries were prepared using previously published primers. Libraries were then pooled at equimolar ratios and sequenced on an Illumina MiSeq Micro 500 nt as paired-end reads. Reads were 2×250 bp with an average depth of ˜33,616 reads. Also included were 8 bp Index reads, following the manufacturer's protocol (Illumina, USA). Raw sequencing reads were filtered by Illumina HiSeq Control Software to generate Pass-Filter reads for further analysis. Index reads were used for sample de-multiplexing. Amplicon sequencing variants (ASVs) were then inferred from the unmerged paired-end sequences using the DADA2 plugin within QIIME2 version 2018.6 (29, 30). Reads were not trimmed. Taxonomy was assigned to the resulting ASVs with a naive Bayes classifier trained on the Greengenes database version using only the target region of the 16S rRNA gene. 13.8. All downstream analyses were performed using family-level taxonomy assignments. Sequencing counts that were present in blank controls were subtracted. Relative abundance, richness, Shannon diversity, and PMA-index were assessed using the Vegan package for R or Microsoft Excel and plotted using R, Prism, and MATLAB. The PMA-index was calculated using relative abundance and was not calculated for any bacterial taxa that was present in fewer than four samples.

By sequencing pairs of matched samples with PMA treatment (PMA-seq) and without PMA treatment (traditional sequencing), one can explore how closely the microbiome compositions obtained from traditional sequencing methods resembled the viable microbiome composition obtained by the disclosed PMA seq.

These tests establish that at each skin site sampled, as compared to traditional sequencing, the PMA-treated samples were less rich (richness, R, is a measure of the number of identifiable bacterial taxa) and less diverse (diversity, H, is measured by the Shannon diversity index). Furthermore, samples that had greater richness in traditional sequencing showed proportionally larger decreases in richness and Shannon diversity with PMA-seq.

These results suggest that, although it appears by traditional sequencing that there is a wide range of richness values at different skin sites (1-30 different taxa), in reality the richness across the skin microbiome at different body sites is relatively similar and low (1-10 different taxa).

To quantify taxon-level PMA-dependent changes, a PMA-index (I_{PMA}) was developed for each bacterial taxon, which is calculated as follows:

$${I}_{PMA} = \frac{{A}_{PMA}}{( {A}_{PMA} ) + ( {A}_{TRAD} )}$$

where A_{PMA} is the relative abundance by PMA-seq and A_{TRAD} is the relative abundance by traditional sequencing. A low PMA-index (below 0.5) indicates that the taxon in question is overrepresented by traditional sequencing, while a high PMA index (above 0.5) indicates that the taxon in question is underrepresented by traditional sequencing. Calculating PMA-indices revealed that the abundances of most bacterial taxa at any given body site are overestimated by traditional sequencing, as most taxa had PMA-index values close to 0.

The three most abundant family-level bacterial families (Propionibacteriaceae, Corynebacteriaceae, and Micrococcaceae) made up 93% of total sequencing reads (96% of PMA-seq reads and 91% of traditional sequencing reads) and demonstrated interesting family level PMA-index patterns. The family Propionibacteriaceae includes a major component of the skin microbiome, C. acnes, which has been shown by traditional sequencing to comprise upwards of 50% of the skin microbiome irrespective of site type (8). PMA-seq revealed that traditional sequencing accurately represents Propionibacteriaceae abundance in sebaceous sites (demonstrated by a PMA-index close to 0.5), but over represents Propionibacteriaceae in moist and dry sites (PMA-indices of 0.2-0.3). Furthermore, Propionibacteriaceae dominated sebaceous sites (accounting for >75% of all viable bacteria in most sebaceous samples) but did not dominate moist or dry sites (their viable abundance did not exceed 50% of all viable bacteria in any of those samples). Bacteria in the family Corynebacteriaceae are also considered main constituents of the skin microbiome, but the present results showed that traditional sequencing overestimates the abundance of Corynebacteriaceae at every skin site except for the nares. For example, traditional sequencing identified a high abundance of Corynebacteriaceae in the popliteal fossa, but PMA seq showed that these reads were largely of inviable origin. Previous studies have demonstrated that Corynebacteria are readily cultured from nasal isolates, which supports the disclosed PMA-seq finding that viable members of this taxon are abundant in the nares but not at most other skin sites. Interestingly, Micrococcaceae were overrepresented by traditional sequencing at every site except for the hair shaft. In the hair shaft, Micrococcaceae were abundant by PMA-seq but almost undetectable by traditional sequencing.

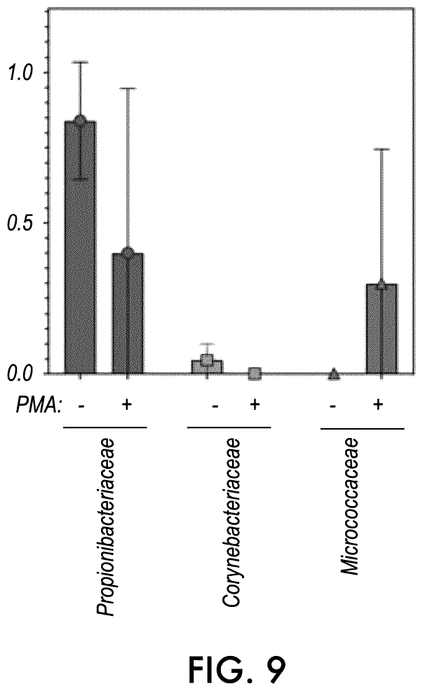

As shown in FIG. 9, the increase in the relative proportion of viable Micrococcaceae detected by PMA-seq corresponds to a decrease in viable Propionibacteriaceae, suggesting that Micrococcaceae may not be detected by traditional sequencing because of the high abundance of DNA from inviable Propionibacteriaceae. These results suggest that most skin sites are colonized by a relatively small number of bacterial families and that different families distinctly colonize different skin sites. The disclosed sequencing did not show an abundance of Staphylococcus, which is often, but not always, detected in the skin microbiome.

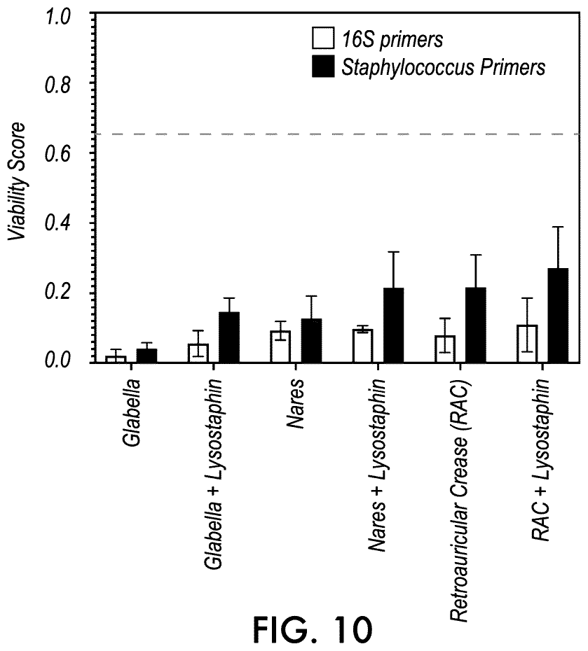

This example therefore evaluated the viability of this genus using PMA-ddPCR with Staphylococcus-specific primers. See FIG. 10. By using lysostaphin prior to DNA isolation, it was ensured that any Staphylococcus present would be sufficiently lysed. Comparisons with universal bacterial 16S primers confirmed that the inclusion of lysostaphin did not significantly change the overall viability scores at the body sites tested. Meanwhile, the Staphylococcus-specific analysis confirmed that like other bacteria, Staphylococcus are also largely inviable on the skin surface. These results reinforce a conclusion that the bulk of the bacterial DNA on the skin surface is from nonviable bacteria.

Finally, we were curious if our findings were human-specific. The spatial distribution of bacterial cells in mouse skin tissue was assessed. Using the universal bacterial FISH probe with tissue from K14-H2B-GFP mice revealed the same bacterial distributions as seen in the human tissues: a high abundance of bacteria in hair follicles (enrichment score of 15.26) with relatively few bacteria on the skin surface (enrichment score of 0.21).

Given that one main function of the densely-packed coat of hair found on mammals like mice is to protect the skin surface from the outside environment, we wondered whether the lack of bacteria on the skin surface was due to their dense fur. Fur does not impact the presence of bacteria on the skin surface—a FISH staining was performed on skin from nude mice (SKH1-Hrhr Elite) and found similar bacterial distributions (follicle-associated enrichment score of 10.79 compared to 1.13 for the skin surface). As a positive control, Escherichia coli cells were applied to dorsal mouse skin tissue after removing it from the animal. This tissue was then processed in the same way as the human and other mouse tissue. FISH staining revealed many bacteria on the surface of these samples, confirming that this technique can be used to reliably visualize bacteria on the skin surface. As a negative control, it was confirmed that a probe encoding the reverse complement of the EUB338 FISH probe (NONEUB338) did not significantly stain the skin surface or follicles.

The viability of bacteria in the mouse skin microbiome was assessed using the disclosed technique (PMA-ddPCR). The PMA-ddPCR-based viability score for mouse skin microbiome sites was similar to the average viability score for human skin sites (0.066 and 0.045 respectively) and was much lower than the viability score for the mouse or human fecal microbiome (0.98 and 0.66 respectively). The results indicate that, despite having distinct skin biology, both humans and mice have an abundance of bacterial DNA on the skin surface that is not associated with viable cells. This observation suggests that the factors leading to this are also not unique to either given

These examples show that traditional 16S rRNA gene amplicon sequencing is not sufficient for analyzing bacterial communities like the skin microbiome, as it leads to overestimation of richness and diversity and can lead to inaccurate assessment of bacterial abundance. The disclosed approach is thus a powerful tool for assessing the viable components of a complex community, and, when coupled with traditional sequencing, can also evaluate how closely the available DNA reflects the viable components within a community. Our results provide an essential step towards a complete understanding of the functional skin microbiome and suggest a more accurate method to evaluate bacterial communities on the skin surface.

It is envisioned that the method disclosed herein can be used on any samples acquired under any condition. It is also envisioned that one of skill in the art will be able to select and use appropriate primers with very little experimentation, if any, required, as part of the ddPCR process, to target one or more strains, species, or genera, as appropriate.

While the invention has been described in detail with reference to certain preferred embodiments thereof, it will be understood that modifications and variations are within the spirit and scope of that which is described and claimed. Since modifications will be apparent to those of skill in this art, it is intended that this invention be limited only by the scope of the following claims.

What is claimed:

1. A method for determining the absolute viability of a bacterial sample, comprising:
splitting a sample containing a plurality of bacterial cells into a first subsample and a second subsample, the first and second subsamples being of equal volume;
treating only the first subsample with PMA, sufficient PMA being added to achieve a target concentration;
incubating the first subsample without exposure to a blue light wavelength for a first period of time;
activating and cross-linking the PMA in the first subsample by exposing the first subsample to blue light wavelengths for a second period of time;
extracting DNA from both the first and second subsamples;
quantifying the extracted DNA from both the first and second subsamples using droplet-digital PCR and gene-specific primers for a target species or strain; and
determining the absolute viability of the original sample by comparing a value of the quantified DNA from the first sample to a value of the quantified DNA from the second sample.

2. The method according to claim 1, wherein the gene-specific primers are universal 16S primers.

3. The method according to claim 1, wherein the gene-specific primers are primers specific to a flagellar gene in Salmonella typhimurium.

4. The method according to claim 1, wherein the first period of time is between 5 and 20 minutes.

5. The method according to claim 4, wherein the second period of time is between 10 and 30 minutes.

6. The method according to claim 1, wherein comparing values of quantified DNA comprises determining a ratio between the value of the quantified DNA from the first sample to the value of the quantified DNA from the second sample.

7. The method according to claim 1, wherein the DNA is extracted using a standard DNA isolation kit.

8. The method according to claim 1, wherein the sample is from a human microbiome, or from a built surface.

9. The method according to claim 8, wherein the sample contains bacteria from human skin, saliva, nasal cavity, or gut.

10. The method according to claim 8, wherein the sample contains bacteria from surface in a hospital, in a house, or in an office building.

11. A method for assessing bacterial community structure changes, comprising:
splitting a sample containing a plurality of bacterial cells into a first subsample and a second subsample, the first and second subsamples being of equal volume;
treating only the first subsample with PMA, sufficient PMA being added to achieve a target concentration;
incubating the first subsample without exposure to a blue light wavelength for a first period of time;
activating and cross-linking the PMA in the first subsample by exposing the first subsample to blue light wavelengths for a second period of time;
extracting DNA from both the first and second subsamples;
sequencing the extracted DNA from both the first and second subsamples using gene-specific primers for a target species or strain and determining an abundance of bacterial species in both the first and second subsamples; and
assessing bacterial community structure changes by comparing the relative abundance of bacterial species in the first and second subsamples.

12. The method according to claim 11, wherein the first period of time is between 5 and 20 minutes.

13. The method according to claim 12, wherein the second period of time is between 10 and 30 minutes.

14. The method according to claim 11, wherein comparing values of quantified DNA comprises determining a ratio between the value of the quantified DNA from the first sample to the value of the quantified DNA from the second sample.

15. The method according to claim 11, wherein the DNA is extracted using a standard DNA isolation kit.

16. The method according to claim 11, wherein the sample is from a human microbiome, or from a built surface.

17. The method according to claim 16, wherein the sample contains bacteria from human skin, saliva, nasal cavity, or gut.

18. The method according to claim 16, wherein the sample contains bacteria from surface in a hospital, in a house, or in an office building.
